(19) United States
(12) Patent Application Publication
(10) Pub. No.: US 2013/0190555 A1
(43) Pub. Date: Jul. 25, 2013

(54) SINGFIT

(71) Applicant: Andrew Tubman, Los Angeles, CA (US)

(72) Inventor: Andrew Tubman, Los Angeles, CA (US)

(21) Appl. No.: 13/692,276

(22) Filed: Dec. 3, 2012

Related U.S. Application Data
(60) Provisional application No. 61/565,775, filed on Dec. 1, 2011.

(51) Int. Cl. A61M 21/00

(52) U.S. Cl. CPC A61M 21/00
USPC 600/28

(57) ABSTRACT

A method of treating patients with special needs to increase or activate brain activity including download one or more songs into the memory of a 4-track play and record device, delivering to a patient via the play and record device audibly the words to be sung in a song just prior to when the words are to be sung, recording the spoken words sung by the patient in response the delivering to the patient step audibly the words to be sung in a song just prior to when the words are to be sung on the play and record device, analyzing the recording and repeating the delivering and record over a period of time and tracking the patient's progress.

### BACKGROUND OF THE INVENTION

Scientific research reveals that singing in general can help reduce pain and stress, boost immune system fortitude, increase the ability to focus and promote brain plasticity and activity. SingFit's handicapped accessible technology is designed for healthcare professionals, and is easily used by patients, family members and caregivers alike. Recent advances in F-MRI/imaging technology as well as the use of Hematological testing have enabled the medical community for the first time, to quantitatively prove the physiological and psychological benefits of singing. The SingFit program enables just about everyone, abled or disabled, to achieve the immediate gratification and clinical benefits of a successful singing experience. Prescribed and directed singing utilizing the SingFit program is a natural, non-invasive activity, delivered in an engaging, simplistic and non-intimidating manner.

In a first time ever clinical study completed at Harvard University in Feb. 2010, it was reported that by using brain imaging technology, ground breaking results were found for rewiring damaged portions of the brain of stroke victims by using interactive singing participation, a.k.a. melodic intonation therapy, to improve and accelerate speech recovery. Essentially this was accomplished by singing in a specific rhythmic manner. By doing so, the brain utilized redundant and alternative neuro-pathways to recover functionality. Other clinical studies recently conducted at leading research universities have also documented the medical and psychological benefits of singing. Some of these benefits include increased neurotransmitter and endorphin generation, elevation of mood, reduction of depressive symptoms, an increase in concentration and memory re-call, pain reduction, and increased immune system fortitude. Scientific studies have shown that singing in general positively impacts the levels of hormones and neuro chemicals responsible for regulating mood, blood pressure and social bonding, including serotonin, cortisol and oxytocin. Furthermore, singing has been proven to help increase immune system strength and respiratory function while decreasing stress, pain and agitation. Imaging studies also reveal that singing can promote cross hemisphere brain activity, which can increase mental neuroplasticity and improve cognitive functioning.

### SUMMARY OF THE INVENTION

The SingFit singing program is a personalized, 4 track software application initially launching on Apple devices such as iPad, iTouch and iPhones. With future design plans for multiple platform use, this user friendly technology enables spontaneous interactive participation for the purpose of providing various special needs populations access to the now proven medical and psychological benefits of singing. This innovative technology also addresses the need of providing access to inexpensive and quality health care to an exploding special needs population all over the world. SingFit is easy-to-use software that utilizes spoken word prompting to feed the words of a song to a singer, right before they need to be sung. This means that a singer does not need to be able to read or even see to sing the words of the song perfectly.

### BRIEF DESCRIPTION OF THE DRAWINGS

FIG. 1 shows the 4-track playback and recording device of the present invention with songs available for download.

FIG. 2 shows the 4-track playback and recording device of the present invention with the lyric coach, guide singer, backing music and input volume tracks displayed on the screen of the 4-track playback and recording device.

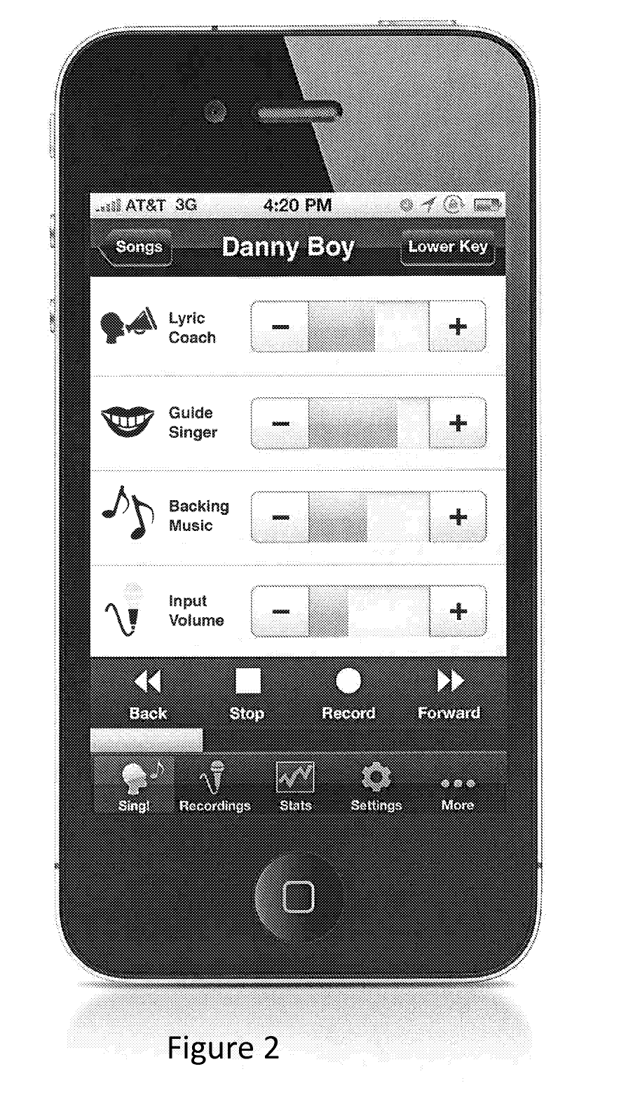

### DETAILED DESCRIPTION OF THE INVENTION

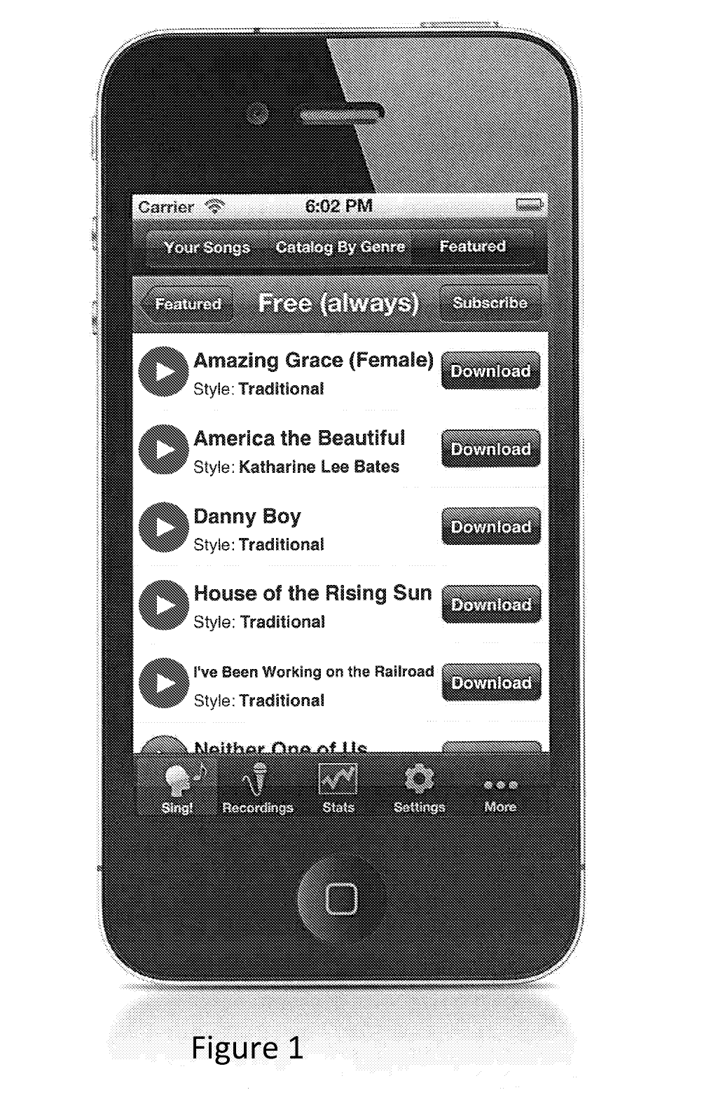

The SingFit program is delivered via a 4-track play and record system. The SingFit delivery methodology provides four tracks of audio in which each track has its own adjustable volume slider/button as shown in FIG. 1 The first track is our easy to use Lyric coach (the spoken words) delivered just prior to when the words are to be sung in the music, the second track is the lead guide singer, the third track is the stereo background music and the fourth track is dedicated for recording while monitoring the volume of one's own voice. The recording track can be turned on and off by the user. All of these tracks are adjustable depending on the participant's choice of comfortable volume levels. Once a participant records their vocal performance, the SingFit program mixes the tracks so that the lyric coach and the guide singer are muted and the participant's recorded voice and the music are mixed into a sharable song file format. A SingFit song requires space between lyrics in order to insert the spoken lyric prompt. In most genres, about 1 in 10 songs has the required space needed for a SingFit song. We have found that rap, hip hop and most pop recorded after the 1980s do not fit SingFit format because of how dense the lyrics are in those genres.

Recordings and “stats” will remain in the device unless legal sharing is specified. Legal sharing consists of the ability to exchange all public domain songs as well as songs that are approved in the SingFit “safe sharing system” in which all recordings are reviewed. by qualified, personnel to insure the song recordings have kept their “intended integrity”. The SingFit software is currently programmed to collect statistics that record “How many minutes sung”, “minutes sung this month”, “minutes sung today”, “number of songs sung”, “number of songs recorded and “number of songs shared”. Depending on the specific population and the purpose of the study, SingFit software can be programed to collect a variety of information, both qualitative and quantitative.

When the SingFit “Lower Key” button is selected, the key of both the music track and the guide singer are instantly transposed down 3 half steps down. The button can be selected while in play mode and the key changes without stopping the song. This feature provides the ability for the participant/assistant to spontaneously match the music to the participant's vocal range or at least change the key of the song enough so that the participant does not need to struggle with trying to sing the majority of notes of the song around the “break” notes in one's voice. Furthermore, this feature helps someone with no vocal training to feel good about their singing experience regardless of vocal range. Other features of the Singh application include: Reverb, stats that can be shared, a mono performance-mix out that allows for group singing, a hide record button feature and assistive instructions and video.

SingFit is intended to promote health and well being by enabling successful singing experiences for practically everyone, including those with Alzheimer's, autism, brain injuries, Down Syndrome, Parkinson's Disease impaired. vision, and other special needs. SingFit's unique Lyric Coach system digitizes a music therapy tool, Lyric Cueing, allowing users to spontaneously participate in joyful, mood elevating and life enhancing singing experiences. Lyric Coach system allows users to enjoy a successful singing experience regardless of their ability to read or see the lyrics to a song.

The SingFit app and a collection of public domain songs are free to download from the Apple App store, and can be used with iPhone, iPad and iTouch hardware. A monthly subscription allows all-you-can-sing access to SingFit's growing catalog of songs featuring a wide range of beloved compositions from multiple musical genres and eras. From Frank Sinatra and Al Green to Bob Marley, Selena, Bonnie Raitt, The Kings of Leon and everybody in between, SingFit delivers an exciting and engaging experience for music lovers.

As shown in FIG. 1, the SingFit App allows a user to download songs and catalog by genre. Songs can be sung and recorded and stored in memory for later play back or analysis. The interface includes a Sing, Recordings, Stats, Settings and More icons.

In the three studies proposed below, in addition to utilizing the existing stats already programed in the application, in-app questionnaires as well as standardized testing will be recorded.

For the studies regarding PTSD and Dementia (both being related to memory), outcomes measures will include but are not limited to; measures of clinical symptoms, systems of care measurements and comparison of memory performance on standardized assessments. Pre and post testing will include: contextual memory test (CMT), SF-36, and PTSD checklist (PCL) to assess memory and quality of life. Post testing will also include completion of the System Usability Scale 2 to assess the feasibility of the application.

To access the SingFit programming, participants utilize a SingFit registered Apple device, such as an iPhone, iPad or iPod and obtain through either the iTunes online store via an individual monthly subscription or through their affiliated institution's custom subscription, Once the initial SingFit software is downloaded onto the participant's Apple device, the participant, or the participant's assistant, will help to download the desired and most engaging songs from the SingFit song library. This library consists of both in-house and sub-contracted re-record music tracks. Public domain songs as well as songs licensed from publishers have been hand picked to fit the SingFit format. The format not only consists of having the various tracks separated for the user to control, but also for therapeutic criteria like, spacing, tempo, range etc.

SingFit is a new and novel way of treating the special needs population. Sing Fit will be used by a user repeatedly over a period of time to increase or activate brain activity. By audibly providing the words to be sung just prior to when the words are to be sung in the music allows the brain to be stimulated and trained to sing. Thus, a method for treating special needs patients involving delivering via a 4-track play and record system audibly the words to be sung just prior to when the words are to be sung in a song has been found to stimulate brain activity. Further, recording the spoken words of the patient while using SingFit and other statistics patients can be analyzed and progress tracked.

The SingFit therapeutic singing program will be implemented in studies with 25 control and 25 placebo participants for three different groups of special needs patients.

In the first study, titled Adjunctive Music Therapy Application with Remote Cognitive Remediation for Improved Memory in Service Members, Veterans with PTSD and mTBI will utilize the SingFit program five times a week, 30-60 minutes each session for 8 weeks.

### The Problem to be Addressed;

This project examined the feasibility of incorporating a novice, commercially available mobile music therapy application to the start and end of occupational therapy psycho-education sessions for improved outcomes for service members with memory deficits due to PTSD and mTBI.

Participants will be initially instructed on how to use the SingFit technology independently and then given instructions by an assistant such as a therapist or supervisor who the participant will see weekly on how to systematically learn songs of participant's choosing. This instruction in general will cover how; to find a song in the SingFit catalogue, download the song from the iTunes app store, connect headphones to the mobile device, open the song on the Singfit “your songs” page, utilize different volume settings, sing and record, then save and listen to their rendition of the song. Though memorization of a given song will be encouraged, the main goal is consistent engagement of at least 30 minutes in the act of singing enabled by the Singfit program. Additional singing sessions will be encouraged if participant found the

Singfit program useful as a coping strategy to reduce PTSD symptomology. Data will be collected weekly over an eight week study through standardized questionnaires filled out by participant, one immediate family member and therapist. Additionally testing can be performed prior to a singing session and over the span of the study data prior and post each singing session will be collected.

Based upon. some initial testing we expect to see results in the range of 70-85% of the 25 Veterans in the control group reporting a reduction in PTSD symptomology and improvement of memory. 100% of the placebo group reporting no change in symptomology. 60-80% of family members reporting observed reduction of PTSD symptomology. 75-90% of assistants reporting an observed reduction of PTSD symptomology. Additionally, we believe that at least 50% of participants that used SingFit will continue to use SingFit and continue to report reduce symptomology in additional voluntary sessions.

In the second study, participants with a diagnosis of early on-set Dementia will utilize the Singfit program four times a week, for 30-60 minutes each session over a six month. period. Participants will be given sufficient assistance to be able to either independently engage in singing using the Singfit technology or the technology was facilitated by an “assistant”. Though memorization of a given song will be encouraged, the main goal was consistent engagement for at least 30 minutes in the act of singing enabled by the Singfit program. Additional singing sessions were encouraged if participant or assistant found the Singfit program useful as a coping strategy to reduce symptoms of Dementia.

### The Problem to be Addressed;

This project examined if a commercially available mobile music therapy application improved outcomes for a general early on-set Dementia population. The testing questionnaires covered reducing agitation and general Dementia symptomology as well as increasing general recall.

Data will be collected weekly over a two-month study through standardized questionnaires filled out by participant, one immediate family member and assistant. Similar instructions will be given to the assistant, that had also been provided in the Veterans study. Additionally testing can be performed prior to a singing session and over the span of the study data prior and post each singing session will be collected.

Based upon some initial testing we expect to see results in the range of 60-80% of the 25 participants experiencing early on-set Dementia, reporting that the SingFit program helped to reduce the symptoms of Dementia and increase recall. 100% of the placebo group reporting no change in symptomology. 65-75% of family members reporting an observed reduction of early on-set Dementia symptomology. Additionally, we believe that at least 40% of participants that used SingFit will continue to use SingFit and continue to report reduce symptomology in additional voluntary sessions.

In the third study, participants will be chosen for this study with a diagnosis of Autism from eight to eighteen years of age who have the ability to vocalize but typically need. a “cue” to initiate an audible word and typically are only able to produce single word utterances. The SingFit program will be utilized for an 8 week period, five times a week, for 30-60 minutes each session. In addition, in the proposed study with the autism spectrum disorders (ASD) population, the SingFit software will record 2 songs of participant's choosing at the beginning and end of each week throughout the 8 week study. These songs will then be shared, analyzed and compared by qualified speech and music therapists to determine various clinical features established in speech and singing development. This analysis will be based on standardized speech and music therapy assessments and focus on developmental goals such as; speech dynamics and expression, length of vocalization and annunciation.

### The Problem to be Addressed;

This project examined if a commercially available mobile music therapy application improved speech output for a sampling of people on the Autism Spectrum. The assumption capitalizes on the inherent musical strengths of those with Autism, and offers activities, singing and technology, that they intrinsically enjoy. It also engages and potentially stimulates a network of brain regions that may be dysfunctional in Autism.

Data will be collected weekly over a two-month study through standardized questionnaires filled out by participant, one immediate family member and the participant's supervising therapist. Similar instructions will be given to the assistant that had also been provided in the Veterans study. Testing will be done prior to a singing session and four of the six tests over the six-month span consisted of collecting data prior and post singing session.

Based upon some initial testing we expect to see results in the range of 80-100% of the 25 participants experiencing speech deficits reporting that the SingFit program helped to increase the amount of time the participant vocalized as well as an increase of the number of words they were able to link together in their speech process. 100% of the placebo group reporting no change in symptomology. 70-80% of family members reporting an. observed increase in appropriate vocalizations. Additionally, we believe that at least 90% of participants that used SingFit will continue to use SingFit and continue to increase their vocalizations.

1. A method of treating patients with special needs to increase or activate brain activity comprising the following steps:
a) download one or more songs into the memory of a 4-track play and record. device;
b) delivering to a patient via the play and record device audibly the words to be sung in a song just prior to when the words are to be sung;
c) recording the spoken words sung by the patient in response the delivering step (b) on the play and record device;
d) analyzing the recording; and
e) repeating the above steps (b-d) over a period of time and tracking the patient's progress.
